(19) United States
(12) Patent Application Publication
(10) Pub. No.: US 2024/0315872 A1
(43) Pub. Date: Sep. 26, 2024

(54) SURGICAL INSTRUMENT WITH INTEGRATED PRESSURE SENSOR FOR OPHTHALMIC SURGERIES

(71) Applicant: Alcon Inc., Fribourg (CH)

(72) Inventor: Satish Yalamanchili, Irvine, CA (US)

(21) Appl. No.: 18/594,209

(22) Filed: Mar. 4, 2024

Related U.S. Application Data
(60) Provisional application No. 63/491,577, filed on Mar. 22, 2023.

(51) Int. Cl. A61F 9/007; A61B 3/16; A61B 17/00; A61B 17/3201; A61B 17/3211; A61B 90/00

(52) U.S. Cl. CPC A61F 9/00736; A61B 3/16; A61B 2017/00199; A61B 17/3201; A61B 17/3211; A61B 2090/064; A61B 2562/0247

(57) ABSTRACT

Embodiments of the present disclosure provide a surgical instrument having a pressure sensor incorporated in, or adjacent to, a distal end of the surgical instrument to enable direct intraocular (IOP) readings of an eye. The surgical instrument may include a hand-held device that is inserted into the eye to perform a surgical task.

### BACKGROUND

During an ophthalmic surgical procedure, such as a vitreoretinal surgery or a phacoemulsification surgery, irrigating fluid is infused into the eye to maintain a relatively consistent intraocular pressure (IOP) while certain materials (e.g., portions of the vitreous or a cataractous lens) are removed from the eye. Measuring IOP is critical during these procedures to prevent the collapse of the eye or a sudden pressure surge (e.g., post-occlusion surge) in the eye.

Currently, during various types of ophthalmic surgical procedures, IOP is estimated based on measurements from pressure sensors disposed external to a patient's eye. For example, in certain examples, a pressure sensor of a surgical console may be utilized to estimate IOP based on pressure(s) within one or more fluid lines operably coupled to the pressure sensor. In certain other examples, a pressure sensor in a handpiece of a surgical tool may be utilized to estimate IOP based on pressure(s) within one or more fluid lines disposed through the handpiece. Because these pressure sensors are located upstream of the eye, IOP control is subject to, among other inaccuracies, a time delay between a pressure event that occurs inside the eye and a sensed pressure change at the surgical console or handpiece end. In addition, the IOP measurements can be prone to inaccuracies introduced by unknown resistance in fluid lines. For example, if the resistance of an irrigation line to the eye changes resistance, it impacts the accuracy of IOP sensing and thus, the efficiency of IOP maintenance. These inaccuracies resulting from measuring IOP away from the eye render external IOP measurement undesirable.

### BRIEF SUMMARY

The present disclosure relates generally to surgical instruments with integrated pressure sensors for ophthalmic surgeries.

Certain aspects provide an endoilluminator, comprising: a handpiece; and a probe coupled to a distal end of the handpiece, the probe comprising an optical fiber and a pressure sensor, wherein: the pressure sensor is positioned near a distal end of the probe, the pressure sensor is configured to be inserted into an intraocular space of an eye, and the pressure sensor is configured to directly sense an intraocular pressure (IOP) associated with the intraocular space of the eye.

Certain aspects provide an instrument for ophthalmic surgery, the instrument comprising: a pressure sensor positioned near a distal end of the instrument, wherein the pressure sensor is configured to: be inserted into an intraocular space of an eye, and directly sense an intraocular pressure (IOP) associated with the intraocular space of the eye.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 illustrates a schematic cross-sectional view of an eye having an endoilluminator with one or more pressure sensors inserted therein for providing direct intraocular pressure (IOP) measurements, according to some embodiments of the present disclosure.

FIG. 2 illustrates a schematic cross-sectional side view of an endoilluminator having one or more pressure sensors integrated at a distal end thereof, according to some embodiments of the present disclosure.

FIG. 3A illustrates a side perspective view of an endoilluminator having one or more

pressure sensors integrated at a distal end thereof, according to some embodiments of the present disclosure.

FIG. 3B illustrates a partial side perspective detailed view of a distal end portion of a tube of the endoilluminator illustrated in FIG. 3A.

FIG. 3C illustrates a cross-sectional front view of a distal end face at the distal end portion illustrated in FIG. 3B.

FIG. 4A illustrates a side perspective view of an endoilluminator having a pressure sensor integrated at a distal end thereof, according to some embodiments of the present disclosure.

FIG. 4B illustrates a partial side perspective detailed view of a distal end portion of a tube of the endoilluminator illustrated in FIG. 4A.

FIG. 4C illustrates a cross-sectional view of the tube of the endoilluminator near the distal end portion illustrated in FIG. 4B.

FIG. 5 illustrates schematic cross-sectional view of an eye having a surgical instrument with a pressure sensor inserted therein for providing direct IOP measurements, according to some embodiments of the present disclosure.

FIG. 6A illustrates a distal end portion of a surgical instrument having a pressure sensor,

according to some embodiments of the present disclosure.

FIG. 6B illustrates another distal end portion of a surgical instrument having a pressure sensor, according to some embodiments of the present disclosure.

FIG. 7 illustrates a schematic diagram of a surgical console and components thereof, according to some embodiments of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

While features of the present invention may be discussed relative to certain embodiments and figures below, all embodiments of the present invention can include one or more of the advantageous features discussed herein. In other words, while one or more embodiments may be discussed as having certain advantageous features, one or more of such features may also be used in accordance with various other embodiments discussed herein. In similar fashion, while exemplary embodiments may be discussed below as device, instrument, or method embodiments it should be understood that such exemplary embodiments can be implemented in various devices, instruments, and methods. As described below, the figures herein each illustrate apparatus and methods for reducing glare and improving surgical visualization of a patient's eye through microscopy.

As used herein, the term “proximal” refers to a location with respect to a device or portion of the device that, during normal use, is closest to the clinician using the device and farthest from the patient in connection with whom the device is used. Conversely, the term “distal” refers to a location with respect to the device or portion of the device that, during normal use, is farthest from the clinician using the device and closest to the patient in connection with whom the device is used. For example, the terms “distal” and “proximal” as used herein may refer to a relative location with respect to an endoilluminator, a microscope, or a portion thereof.

As used herein, the term “about” may refer to a +/−10% variation from the nominal value. It is to be understood that such a variation can be included in any value provided herein.

According to embodiments of the present application, a pressure sensor is incorporated in, or adjacent to, a distal end of a surgical instrument to provide direct intraocular (IOP) readings from an anterior chamber or a posterior chamber of an eye. The surgical instrument may be a hand-held device that may be inserted into the eye to perform a surgical task.

In some embodiments, the pressure sensor is incorporated in, or adjacent to, a distal end of an illumination device (e.g., an endoilluminator) to provide IOP readings from the anterior chamber or the posterior chamber of the eye.

In some embodiments, the pressure sensor is incorporated in, or adjacent to, a distal end of another surgical instrument (e.g., a phacoemulsification probe, a vitrectomy probe, a tissue manipulation device, or another surgical instrument instead of an illumination device), which may be inserted into or disposed adjacent to the anterior chamber or the posterior chamber of the eye.

In some embodiments, examples of other surgical instruments in which one or more pressure sensors may be incorporated may include, but are not limited to, a trocar cannula, an infusion cannula, and other similar devices.

Other examples of surgical instruments into which one or more pressure sensors may be incorporated may include, but are not limited to, a cutting probe, a vitrectomy probe, a phacoemulsification probe, a laser probe, an ablation probe, a vacuum probe, a flushing probe, scissors, forceps, a spatula, a hook, a Sinskey hook, a depressor, a side-port blade, a side-port knife, a knife for micro incision coaxial surgery (MICS), an endoscopic visualization probe, other ophthalmic devices, and/or combinations thereof. The surgical instrument may have configurable operating settings. An operating setting is a parameter with a value that may be selected by a user (e.g., a surgeon, medical technician, or other medical professional).

In some embodiments, the pressure sensor is incorporated in, or adjacent to, a distal end of a surgical instrument and is coupled to controller circuitry for identifying a pressure-related event, such as an increase or decrease in pressure, high pressure, low pressure, or globe rupture or collapse, in the eye.

In some embodiments, the pressure sensor is configured to be inserted into the eye and interface (e.g., be in direct or immediate contact with) with the intraocular space, such as with the materials (e.g., fluids) in the eye to directly sense the IOP associated with the intraocular space in the eye. Therefore, directly sensing the IOP refers to sensing being performed by a pressure sensor while interfacing with the intraocular space.

In some embodiments, direct IOP readings/measurements (and/or information derived from the pressure sensor readings) generated by the pressure sensor can be visually displayed (for example, on a stand-alone display, a heads-up display, an in-microscope display, or a display integrated into a surgical tray or console) and/or audibly announced. In some embodiments, alarms and/or safety measures may be activated based on the direct IOP readings/measurements. Furthermore, the direct IOP readings/measurements may be used, in some embodiments, in a feedback control loop to control the rate of infusion and/or aspiration during anterior segment or posterior segment surgical procedures. n some embodiments, the direct IOP readings/measurements can be used to control and/or maintain IOP in the eye, for example, by adjusting the rate of infusion and/or aspiration during ophthalmic (e.g., anterior segment or posterior segment) surgical procedures.

In some embodiments, examples of pressure sensors may include, but are not limited to, microelectromechanical system (MEMS) pressure sensors, piezoelectric pressure sensors, potentiometric pressure sensors, inductive pressure sensors, strain gauge pressure sensors, capacitive pressure sensors, fiber-based pressure sensors, other miniature pressure sensors, and/or any combination thereof.

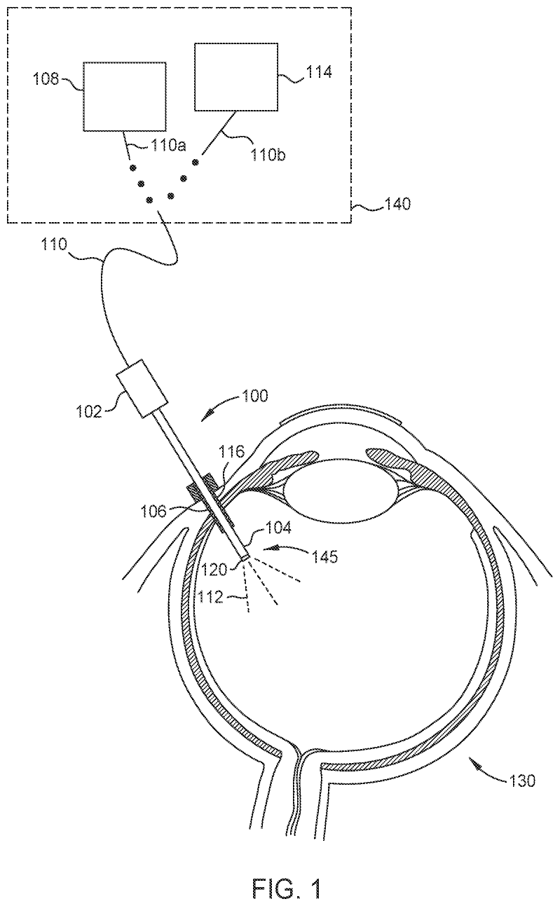

Turning now to FIG. 1, a schematic cross-sectional view of an eye 130 is illustrated with an endoilluminator 100 inserted therein, according to some embodiments of the present disclosure. The endoilluminator 100 comprises one or more miniature pressure sensors 120 for providing direct measurements of IOP from within the eye 130. In addition to providing direct IOP measurements, the endoilluminator 100 is also configured to provide illumination to the intraocular space of the eye 130 to facilitate a surgical procedure therein.

As illustrated in FIG. 1, the endoilluminator 100 includes a handpiece 102 and a shaft or tube 104. The handpiece 102 is coupled to a proximal end of the tube 104. In some embodiments, the handpiece 102 provides a user (e.g., an ophthalmic surgeon) with a graspable portion of the endoilluminator 100 to enable the user to manipulate the depth and location of the tube 104 within the eye 130, to direct light 112 propagated from a distal end 145 of the tube 104, and to position the pressure sensor(s) 120 within the eye 130.

In some embodiments, the tube 104 is a substantially hollow metal shaft or hypodermic tubing configured to be inserted into the eye 130 via an entry cannula 106 disposed through a sclerotomy 116 in the eye 130. In some embodiments, the tube 104 is formed of stainless steel, aluminum, nitinol, other alloys, and/or other suitable surgical-grade metal materials. In some examples, the tube 104 is fixedly coupled to the handpiece 102. In other examples, the tube 104 is rotatable relative to the handpiece 102. That is, the handpiece 102 includes a portion having a circular cross section configured to be rotatably coupled to the tube 104 such that the user can rotate the tube 104 to adjust the incidence of light 112 on the eye 130 and/or adjust the position of the pressure sensor(s) 120 in the eye 130. In another example, the handpiece 102 is fixedly coupled to the tube 104 such that the user can rotate the tube 104 by rotating the handpiece 102. Although the tube 104 of FIG. 1 is illustrated as a straight shaft, other embodiments may include a tube having other shapes. For example, a portion of the tube 104 may be curved or bent to provide light 112 to regions of the eye 130 that would otherwise be difficult to illuminate with a straight tube 104. In some embodiments, the endoilluminator 100 and its components may be an instrument kit for use in ophthalmic surgery.

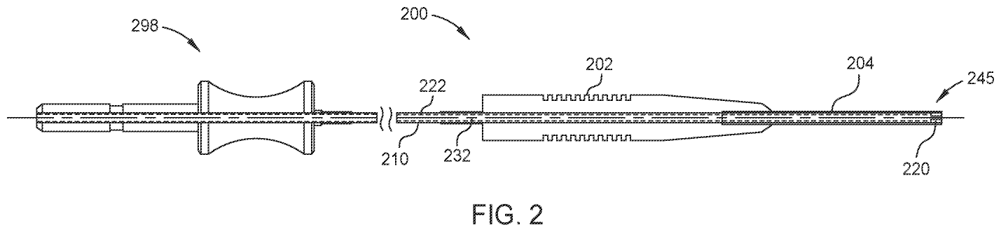

In some embodiments, the endoilluminator 100 is configured to house a single optical fiber 110a or multiple (e.g., a bundle of) optical fibers 110a which are optically coupled to a light source 108 at a distal end of the fiber(s). In some embodiments, the one or more optical fibers 110a may be directly or indirectly attached to an interior chamber of the endoilluminator 100 through the handpiece 102 and the tube 104. The one or more optical fibers 110a are (see, e.g., FIGS. 2, 3A-3C, and 4A-4C) configured to direct light 112 out of the distal end 145 of the tube 104. For example, the distal end 145 of the tube 104 comprises one or more openings through which the distal end(s) of the optical fiber(s) 110a are able to propagate the light 112 into the eye 130. In some embodiments, the optical fiber(s) 110a may include an optical fiber array (e.g., a plurality of optical fibers in regular linear arrangement or 2-dimensional pattern arrangement) and/or one or more multi-core optical fibers (e.g., single-mode (SM) or multi-mode (MM) fibers with multiple cores). In particular, the hollow portion of the tube 104 includes an interior compartment configured to house the optical fiber(s) 110a. The optical fiber(s) 110a may include one or more of a polarization maintaining fiber(s), a polarizing fiber(s), and/or any other light fiber(s) suitable for transmission of light.

In some embodiments, the endoilluminator 100 is further configured to house the one or more pressure sensors 120 and electrical wires (or cables) 110b required for powering the pressure sensor(s) 120 and/or exchanging (e.g., transmitting and receiving) data/signals with, for example, a controller 114 (e.g., having control circuitry). In some embodiments, the electrical wire(s) 110b for the pressure sensor(s) 120 may be, along with the optical fibers 110a, directly or indirectly attached to the interior chamber of the endoilluminator 100 through the handpiece 102 and tube 104. In some embodiments, the electrical wire(s) 110b for the pressure sensor(s) 120 are longitudinally disposed along the direction of the optical fibers 110a in the handpiece 102 and tube 104.

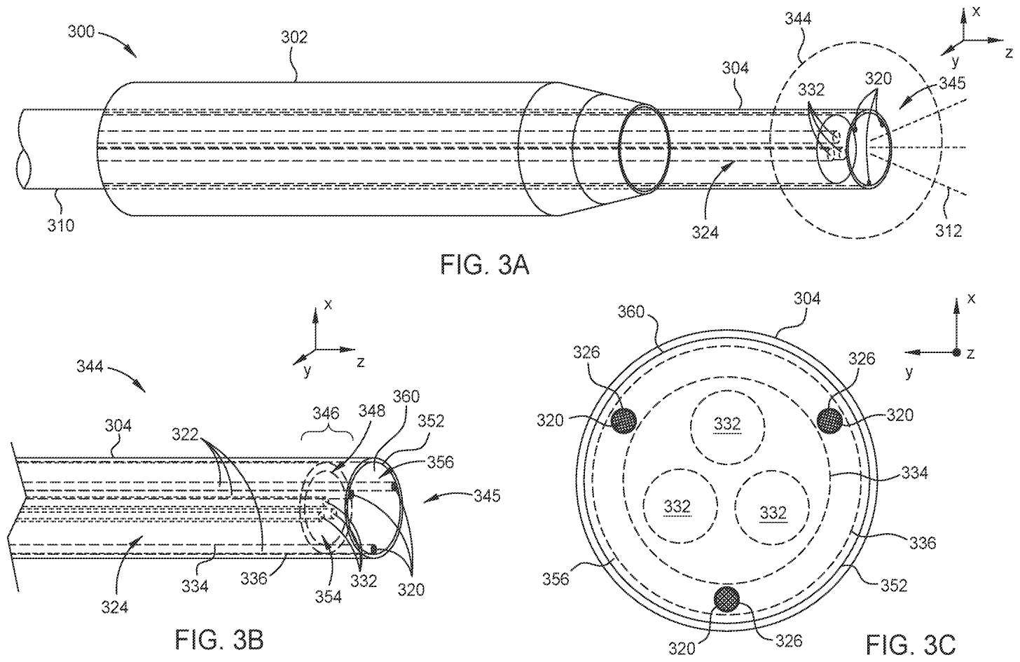

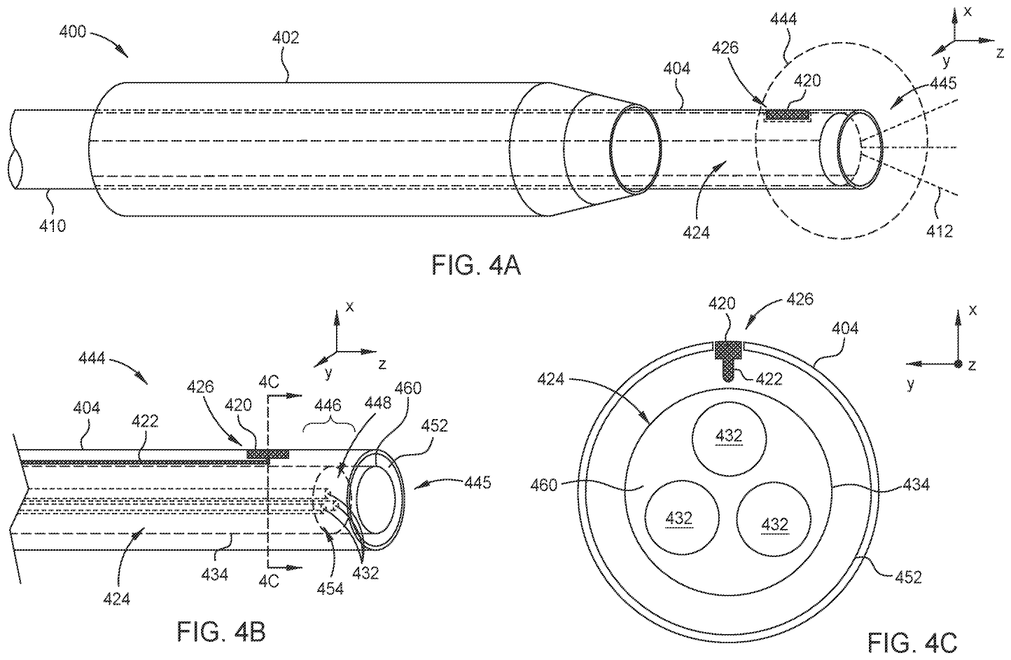

In some embodiments, the pressure sensor(s) 120 may be exposed (e.g., to an external environment outside of the endoilluminator 100) via one or more openings at the distal end 145 of the tube 104, and configured to take direct pressure (e.g., IOP) measurements from within the eye 130 when the endoilluminator 100 is inserted into the eye 130. For example, the distal end 145 of the tube 104 comprises one or more openings through which the miniature pressure sensor(s) 120 may be in direct contact with the fluid(s) inside the eye 130. In some embodiments, the one or more openings for the pressure sensor(s) 120 (as shown in FIGS. 3A-3C) are positioned adjacent to openings through which the distal ends of the one or more optical fibers 110a are able to propagate light 112 into the eye 130. In some embodiments, the one or more openings for the pressure sensor(s) 120 (as shown in FIGS. 4A-4C) are formed in a sidewall of the tube 104 near the distal end 145.

In some embodiments, the endoilluminator 100 is operably coupled to and/or in communication with a surgical console 140 through a cable 110, which is configured to sheath the one or more optical fibers 110a and one or more electrical wires 110b. As illustrated in FIG. 1, the endoilluminator 100's handpiece 102 is coupled to a distal end of the cable 110, while a proximal end of the cable 110 interfaces with the surgical console 140. The one or more optical fibers 110a and one or more electrical wires 110b for the miniature pressure sensor(s) 120 are routed from the endoilluminator 100, through the optical cable 110, and to a light source 108 and the controller 114 in the surgical console 140, respectively.

In the illustrated example, the surgical console 140 includes the light source 108 and the controller 114. However, it should be noted that, in some embodiments, the light source 108 and controller 114 may be located outside of the surgical console 140. In some embodiments, in addition to housing the light source 108 and controller 114, the surgical console 140 may be configured to interface with and drive other surgical instruments and systems, which may include, but are not limited to, ophthalmic probes of a variety of probe types, including laser probes (e.g., picosecond infrared laser probes, femtosecond laser probes), vitrectomy probes, phacoemulsification probes, flap cutters, and other ophthalmic surgical tools. In operation, the surgical console 140 may function to assist the user in performing various ophthalmic surgical procedures, such as vitrectomy, phacoemulsification, cataract surgery, LASIK (Laser-Assisted In Situ Keratomileusis), and similar procedures.

In some embodiments, the light source 108 may be configured to generate and drive light 112 into proximal entry-point(s) of the optical fiber(s) 110a, which may in turn propagate the light 112 to the distal end 145 of the tube 104. In some embodiments, the light source 108 may comprise high brightness phosphor-based white LED(s) and/or RGB LED(s). In some embodiments, more than one light source 108 may be provided to simultaneously provide light 112 to the endoilluminator 100 through an optical coupling. In some embodiments, a single light source 108 can be shared among two or more illumination components, for example through a free-space or fiber splitter. In some embodiments, the light source 108 may include a xenon, mercury vapor, halogen, and/or other light source(s) suitable for ophthalmic surgery. It should be noted that, in some embodiments, the light source 108 may not be external to the handpiece 102. For example, in certain embodiments, the handpiece 102 may contain the light source 108 within its housing or structure.

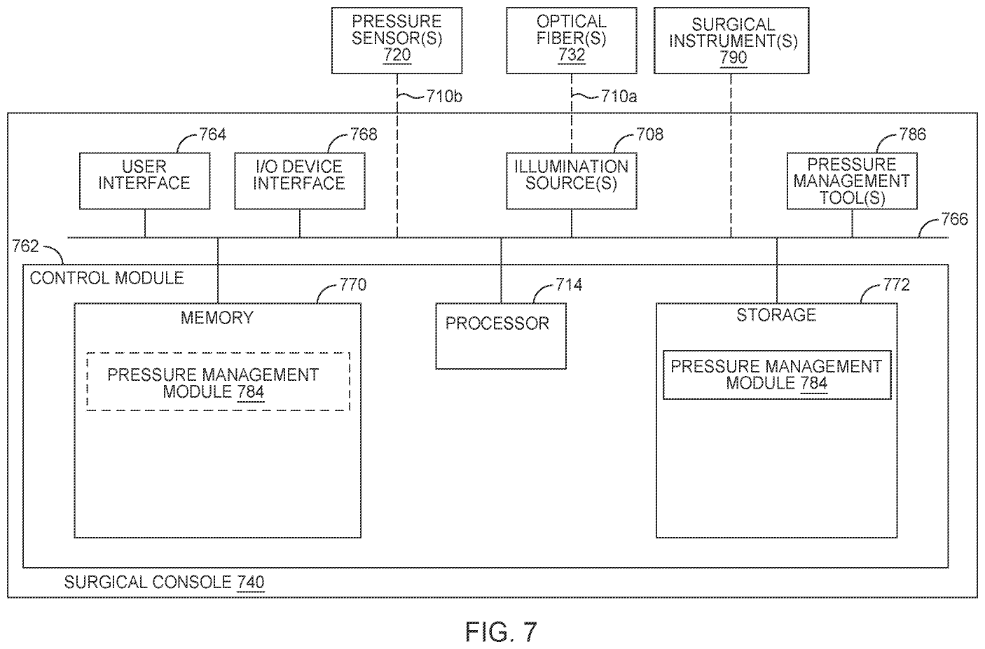

In some embodiments, the controller 114 may be configured to receive data/signals from the pressure sensor(s) 120 via the electrical wiring cable(s) 110b. These data/signals, for example, may correspond to readings of fluidic pressure (e.g., direct IOP measurements) in the eye 130. The controller 114 is also configured to send output signals to other components (e.g., infusion and aspiration lines, valves, and pumps, etc.) coupled to the surgical console 140 to control and/or maintain a desired IOP of the eye 130 during surgery based on the direct IOP measurements. Details of the controller 114 will be described with reference to FIG. 7 below. It should be noted that, in some embodiments, the controller 114 may not be external to the handpiece 102. For example, in certain embodiments, the handpiece 102 may contain the controller 114 within its housing or structure. In certain embodiments, the controller 114 or a separate power source may be configured to provide power to the pressure sensor(s) 120 via the electrical wiring cable(s) 110b.

According to embodiments of the present disclosure, the pressure sensor(s) 120 integrated at the distal end 145 of the tube 104 may be inserted into the eye 130 to provide direct IOP measurements of the eye 130, for example, during vitreoretinal and cataract surgeries. It is important and necessary to monitor IOP within the eye 130 during an ophthalmic surgery such as a vitreoretinal or cataract surgery. Lack of control over IOP may impair the effectiveness or ease of the procedure, and in certain cases may result in damage to tissue. For example, under-pressurizing the intraocular region may result in a collapse of the globe of the eye 130 with concomitant tissue damage. Conversely, over-pressuring the intraocular space may also result in damage to the sensitive retinal, optic nerve, or corneal tissue. Occasionally, in certain instances, it may be desirable to apply controlled high pressure for a brief time period, for example, to staunch bleeding in the intraocular space.

The integration of the pressure sensor(s) 120 positioned at the distal end 145 of the tube 104 enables improved IOP control, thereby maintaining a stable anterior or posterior chamber. The integration eliminates the sensitivity to external factors during closed-loop control of IOP due to the direct measurement capability. Moreover, integrating the pressure sensor(s) 120 at the distal end 145 of the tube 104 allows for faster response to unfavorable pressure-related events occurring during surgery. For example, because the pressure sensor(s) 120 provides direct IOP measurements in the eye 130, as opposed to providing pressure estimates upstream to the eye (e.g., at a surgical console), the endoilluminator 100 effectively eliminates inaccuracies and inefficiencies that would otherwise be introduced by time delays between a pressure-related event that occurs inside the eye 130 and the sensed upstream change in pressure, and/or any inaccuracies caused by resistance in fluid lines.

For vitreoretinal and similar surgeries, since endoillumination is utilized throughout the surgery and since the pressure sensor(s) 120 are integrated into the distal end of the tube 104 of endoilluminator 100, according to the embodiments of the present application, the endoilluminator 100 provides the user a means for receiving direct IOP measurements of the eye 130 throughout the surgery. In addition, because the electrical wires 110b for the pressure sensor(s) 120 are disposed longitudinally with the optical fibers 110a, the integration of the pressure sensor(s) 120 into the endoilluminator does not significantly increase the overall size of the endoilluminator 100. Thus, the endoilluminator 100 is able to maintain a relatively small incision in the eye 130 for insertion.

Additionally, for cataract and similar surgeries, the pressure sensor(s) 120 directly sensing IOP in the eye 130 eliminates the need to take into account wound constriction characteristics near an incision site and/or surgical instrument materials when measuring pressure. Such wound constriction characteristics and instrument materials can greatly impact pressure readings when the pressure is determined indirectly, thus leading to inaccurate assessment of IOP. For example, different materials for irrigation and/or aspiration lines of a surgical instrument may have varying fluidic resistances, thereby leading to differing readings of IOP.

Furthermore, unlike systems where pressure sensing elements are operably coupled with an operating infusion line or aspiration line, the endoilluminator 100 having the integrated pressure sensor(s) 120, according to embodiments of the present disclosure, is not subject to pressure reading inaccuracies as caused by the proximity of pressure sensing elements to an operating infusion line or aspiration line, or by debris clogging the corresponding channel.

By directly monitoring IOP in real time, the user (e.g., a surgeon) and/or surgical console may better control one or more pressure management tools and/or surgical tools (e.g., valves on one or more of the infusion and/or aspiration lines) to maintain IOP within a predetermined range of values. For example, the surgeon and/or surgical console may control IOP by adjusting the irrigation source pressure to a level appropriate for the combination of other settings used (aspiration rate, vacuum limit, tip, sleeve, etc.) for the ongoing procedure. The surgeon and/or the surgical console may evaluate and establish a certain IOP level based on his/her experience with a particular instrument and/or stored values, procedures, programs and applications, respectively.

According to particular embodiments of the present disclosure, the pressure sensor(s) 120 may take 3 milliseconds (msecs) or less to detect a pressure change in the eye 130. Detecting the pressure change within the first 3 msecs (milliseconds), or less, allows the surgical console 140 to provide a fast response to compensate for the pressure change, thereby greatly reducing the risk of damage to the eye 130 and even rupture or collapse. In some embodiments, algorithms that are used in prior predicate devices, including all the various algorithms for the Active Surge Mitigation and/or Active Sentry System developed by Alcon, Inc. of Fort Worth, Texas, may be readily applicable for pressure management (e.g., pressure compensation and control) with the systems and methods described herein.

FIG. 2 illustrates a schematic cross-sectional side view of an exemplary endoilluminator 200 having one or more miniature pressure sensors 220 integrated at a distal end of the endoilluminator 200, according to some embodiments of the present disclosure. In some embodiments, the endoilluminator 200 may substantially correspond to the endoilluminator 100 in FIG. 1. For example, the endoilluminator 200 may include a handpiece 202, a tube 204, a cable 210, and pressure sensor(s) 220 which may substantially correspond to the handpiece 102, tube 104, cable 110, and pressure sensor(s) 120, respectively, of the endoilluminator 100 in FIG. 1. Thus, the details of the handpiece 202, tube 204, cable 210, and pressure sensor(s) 220 are omitted for brevity.

As illustrated in FIG. 2, one or more optical fibers 232 (large dashed line) are disposed through the tube 204, handpiece 202, and cable 210 of the endoilluminator 200 for propagating illumination light, as generated by an illumination light source, to a distal end 245 of the tube 204 where the optical fibers 232 terminate. As further shown, the pressure sensor(s) 220 are positioned at the distal end 245 to facilitate direct measurement of IOP inside of a patient's eye. Accordingly, in some embodiments, the pressure sensor(s) 220 may be operably coupled to the one or more electrical wires 222 (small dashed lines) at the distal end 245 of the tube 204. Along with the optical fibers 232, such electrical wire(s) 222 may extend through the tube 204, handpiece 202, and cable 210 for coupling with a controller, such as a controller integrated with a surgical console, to facilitate operation of the miniature pressure sensor(s) 220. As illustrated in FIG. 2, an adaptor 298, such as a plug, may be used to couple the cable 210 from the endoilluminator 200 to the surgical console.

The increase in diameter of the tube 204, handpiece 202, and/or cable 210 to accommodate the electrical wire(s) 222 may be minimal relative to conventional endoilluminators, since such components are already configured to have one or more optical fibers and/or electrical wires integrated therein for driving probe function. In one particular example, in a handheld illumination device (e.g., an endoilluminator), the total diameter increase of a cable to accommodate such electrical wire(s) 222 for the pressure sensor(s) 220, the optical fiber(s) 232, and other electrical wire(s) of the endoilluminator 200 may be less than or equal to 0.5 millimeters (mm). In some embodiments, the integration minimizes additional electrical wiring with substantially zero increase in diameter.

FIGS. 3A, 3B, and 3C illustrate various views of an endoilluminator 300 having one or more miniature pressure sensors 320 integrated at a distal end thereof, according to some embodiments of the present disclosure.

FIG. 3A illustrates a side perspective view of the endoilluminator 300, according to some embodiments of the present disclosure. As illustrated in FIG. 3A, the endoilluminator 300 includes a handpiece 302 and a shaft or tube 304. The handpiece 302 is coupled to a proximal end of the tube 304. In some embodiments, the handpiece 302 and tube 304 may substantially correspond to the handpiece 102 and tube 104, respectively, in FIG. 1. In some embodiments, the handpiece 302 and tube 304 maybe formed from one or more surgical-grade metallic materials, such as, for example, stainless steel, titanium, nitinol, aluminum, or platinum.

In the illustrated example, the endoilluminator 300 includes a bundle 324 extending through the endoilluminator 300. The bundle 324 includes one or more optical fibers 332 and electrical wires (e.g., electrical wires 322 as illustrated in FIG. 3B) for the pressure sensors 320 disposed longitudinally (e.g., in the z-direction) within the handpiece 302 and tube 304.

The optical fibers 332 are configured to receive light 312 from one or more light sources (e.g., the light source 108 in FIG. 1) and propagate the received light 312 to a distal end 345 of the tube 304 where light 312 is emitted. Each of the optical fibers 332 may include a cladding or may be unclad. In some embodiments, one or more of the optical fibers 332 are sapphire fibers or other optically transmissive material. In some embodiments, one or more of the optical fibers 332 may have uniform material compositions along a length of the optical fibers 332. In other embodiments, one or more of the optical fibers 332 may have a first region having a first material composition and a second region having a second material composition where the first material composition and the second material composition are distinct from one another in one or more aspects. In some embodiments, one or more of the optical fibers 332 may be a single-core fiber. In other embodiments, one or more of the optical fibers 332 may be a multi-core fiber.

In some embodiments, the light source(s) may generate unpolarized light 312, which is received and propagated the optical fibers 332 of by the endoilluminator 300. In such embodiments, the optical fibers 332 may polarize the received light 312 and maintain the polarization while propagating the light 312 to the distal end 345 of the tube 304 where the light 312 is emitted.

In some embodiments, the light source(s) may provide polarized light 312, which is received and propagated by the optical fibers 332 of the endoilluminator 300. In such embodiments, the optical fibers 332 can maintain and/or change the polarization of the received light 312. In certain examples, the optical fibers 332 may receive linearly polarized light 312 from the light source(s). In certain examples, the optical fibers 332 may be configured to circularly or elliptically polarize the received light 312, and maintain the circular polarization of the light 312 as it is propagated through the endoilluminator 300. Alternatively, the optical fibers 332 may be configured to maintain the linear polarization of the light 312 received from the light source(s) as it is propagated through the endoilluminator 300 to the distal end 345 of the tube 304 where the light 312 is emitted.

In some embodiments, each of the optical fibers 332 may have a diameter of between about 0.1 mm and about 1.0 mm, such as between about 0.2 mm and about 0.8 mm, such as between about 0.3 mm and about 0.7 mm, such as between about 0.4 mm and about 0.6 mm, such as about 0.5 mm, although other suitable dimensions are also contemplated. In the illustrated example, the endoilluminator 300 includes three optical fibers 332, although the endoilluminator 300 may include any suitable number (fewer or more than three) of optical fibers 332, for example, to provide desired illumination characteristics.

In some embodiments, the pressure sensors 320 are situated at the distal end 345 of the tube 304, and connected to the electrical wires 322 positioned in the longitudinal direction (e.g., the z-direction) along the optical fibers 332 in the bundle 324. In some embodiments, the pressure sensors 320 may substantially correspond to the pressure sensors 120 in FIG. 1.

FIG. 3B is a partial side perspective detailed view of a distal end portion 344 of the tube 304 shown in FIG. 3A. It is noted that the distal end portion 344 of the tube 304 may also be the distal end portion of the endoilluminator 300, and the distal end 345 of the tube 304 may also be the distal end of the endoilluminator 300. As illustrated in FIG. 3B, the bundle 324 includes the optical fibers 332 and electrical wires 322 for the pressure sensors 320.

As illustrated in FIG. 3B, a lens (or window) 360 is disposed in an opening 352 within the distal end 345 of the tube 304. The optical fibers 332 each terminate at or substantially near an interface 346 of the bundle 324 and the lens 360. The interface 346 and/or lens 360 may be configured to facilitate propagation of a desired illumination pattern from the optical fibers 332 toward a target site, such as a target site within an eye of a patient. In some embodiments, the pressure sensors 320 extend beyond the interface 346 and through the opening 352 to sense the IOP associated with the intraocular space in the eye.

In some embodiments, a distal end 348 of the bundle 324 may abut a proximal end face 354 of the lens 360 with positive pressure at the interface 346. In other embodiments, one or more optically transmissive elements or materials may be situated at the interface 346 between the distal end 348 and the lens 360. In some implementations, the lens 360 may be a GRIN (Gradient-index) lens, a spherical lens, or an aspherical lens. In still other implementations, the lens 360 may be a group of lenses formed of optically clear material.

The lens 360 may include one or more lenses formed from a visibly transparent glass or ceramic. For example, the material used to form the one or more lenses of the lens 360 may include fused silica, borosilicate, or sapphire. In some implementations, the lens 360 may include a single-element cylindrical GRIN rod lens that is operable to receive one or more illumination lights 312 from the optical fibers 332 and relay the received illumination lights 312 toward the distal end 345 of the tube 304. In some instances, the distal end 345 of the tube 304 may also correspond to the distal end of the lens 360. In other instances, a protective window may be disposed between the distal end of the lens 360 and the distal end 345 of the tube 304. In still other implementations, the window may extend beyond the distal end 345 of the tube 304.

As described above with reference to FIG. 3A, the pressure sensors 320 are disposed at the distal end 345 of the tube 304. More particularly, in the example of FIG. 3B, the pressure sensors 320 are disposed through a distal end face 356 of the lens 360, thus exposing the pressure sensors 320 to an external environment (e.g., external to the endoilluminator 300). Accordingly, in FIG. 3B, the electrical wires 322 extend beyond the distal end 348 and the interface 346, and through the lens 360, to connect with the pressure sensors 320. In such examples, the electrical wires 322 and pressure sensors 320 may be disposed through one or more features formed in the lens 360, such as openings 326 described below with reference to FIG. 3C. By exposing the pressure sensors 320 to the external environment, the pressure sensors 320 can be in contact with the materials (e.g., fluid(s)) in the eye of the patient to take direct IOP measurements during ophthalmic surgical procedures. In certain examples, the pressure sensors 320 may interface (e.g., be in direct or immediate contact with) with the intraocular space, such as with materials in the eye to directly sense the IOP associated with the intraocular space in the eye.

FIG. 3C illustrates a cross-sectional front view of the distal end 345 of the tube 304 of the endoilluminator 300 illustrated in FIG. 3B. As shown, the pressure sensors 320 are each disposed within (e.g., embedded in) an opening 326 in the lens 360, and are exposed on the distal end face 356 of the lens 360. The openings 326 may extend through an entire thickness (e.g., in the z-direction) of the lens 360 to facilitate external exposure of the pressure sensors 320. In such embodiments, the electrical wires 322 may extend through the openings 326 in the lens 360 to connect with the pressure sensors 320. Thus, unlike the optical fibers 332, the pressure sensors 320 are not covered or shielded by the lens 360, and are configured to be in direct contact with the fluid(s) in the eye to provide direct IOP measurements.

In some embodiments, the pressure sensors 320 are not disposed in or through the lens 360, and the lens 360 only covers a portion of the bundle 324 having the optical fibers 332. In such embodiments, the pressure sensors 320 may be disposed in a space between the lens 360 and the tube 304. In such embodiments, the pressure sensors 320 are exposed at the distal end 345 of the tube 304, and the electrical wires 322 extend beyond the interface 346 in the space between the lens 360 and tube 304 to connect with the pressure sensors 320. In such embodiments, a filler material may be used to fill in the space between the lens 360 and tube 304, such as an adhesive, and to hold the pressure sensors 320 and electrical wires 322 in place.

As further illustrated in FIG. 3C, the optical fibers 332 are disposed in an optional sleeve 334. In some embodiments, the sleeve 334 may include a filler material to fill in the space not occupied by the optical fibers 332. In some embodiments, the pressure sensors 320 and electrical wires 322 are disposed in a second optional sleeve 336 disposed around or adjacent to the sleeve 334. In some embodiments, the sleeve 336 may also include a filler material to fill in the space not occupied by the pressure sensors 320 and electrical wires 322. In some embodiments, the optical fibers 332 and pressure sensors 320 along with the electrical wires 322 may be disposed in a single sleeve, which may include a filler material to fill in the space not occupied by the optical fibers 332, pressure sensors 320, and electrical wires 322. In some embodiments, the optical fibers 332 and/or the pressure sensors 320 are coupled to a sidewall of the tube 304, or are freely disposed within the tube 404 without a sleeve.

Although three optical fibers 332 are shown in the illustrated example in FIGS. 3B and 3C, the scope of the disclosure is not so limited. Rather, in other implementations, the endoilluminator 300 may include fewer optical fibers 332, while other implementations may include more than three optical fibers 332. In some implementations, the endoilluminator 300 may include two, four, or more optical fibers 332, and, in some examples, the optical fibers 332 may form a 2×2 array.

Further, although three pressure sensors 320 are shown in the illustrated example in FIGS. 3B and 3C, the scope of the disclosure is not so limited. Rather, in other implementations, the endoilluminator 300 may include fewer pressure sensors 320, while other implementations may include more than three pressure sensors 320. In some implementations, the endoilluminator 300 may include one, two, four, or more pressure sensors 320, for example, to provide direct IOP measurements.

In certain embodiments, the tube 304 may have a diameter of about 1.5 mm or less, such as about 0.8 mm or less, although other suitable dimensions are also contemplated. In certain embodiments, the diameter of the tube 304 may be about 0.5 mm or less. Generally, the tube 304 may be sized to easily pass through a corresponding entry cannula, such as a trocar cannula, and into an intraocular space of a patient's eye. In certain embodiments, the diameter of the tube 304 corresponds to a gauge size of about 21-Ga (Gauge), 22-Ga, 23-Ga, 24-Ga, 25-Ga, 26-Ga, 27-Ga, 28-Ga, 29-Ga, 30-Ga, or the like. In certain embodiments, the diameter of each miniature pressure sensor 320 may be between about 0.25 to 1.55 mm, although other suitable dimensions are also contemplated.

Accordingly, the surgeon and/or the surgical console is provided a means for directly measuring IOP inside the eye, such that necessary adjustments (e.g., in response to a sudden change in IOP during surgery) can be made to more efficiently and safely control and/or maintain IOP in the eye.

FIGS. 4A, 4B, and 4C illustrate various views of an endoilluminator 400 having a miniature pressure sensor 420 integrated at a distal end thereof, according to some embodiments of the present disclosure.

Turning to FIG. 4A, a side perspective view of the endoilluminator 400 is shown, according to some embodiments of the present disclosure. As illustrated, the endoilluminator 400 includes a handpiece 402 and a shaft or tube 404. The handpiece 402 is coupled to the proximal end of the tube 404. In some embodiments, the handpiece 402 and tube 404 may substantially correspond to the handpiece 102 and tube 104, respectively, in FIG. 1. In some embodiments, the handpiece 402 and tube 404 may be formed from one or more surgical-grade metallic materials, such as, for example, stainless steel, titanium, nitinol, aluminum, or platinum.

Similar to other endoilluminators described herein, the endoilluminator 400 includes a bundle 424 that extends through the endoilluminator 400. The bundle 424 includes one or more optical fibers 432 and electrical wire(s) 422 (as illustrated in FIG. 4B) for the pressure sensor 420 disposed longitudinally (e.g., in the z-direction) within the handpiece 402 and tube 404.

The optical fibers 432 are configured to receive light 412 from one or more light sources (e.g., the light source 108 in FIG. 1) and propagate the received light 412 to a distal end 445 of the tube 404 where the light 412 is emitted. In some embodiments, the optical fibers 432 may substantially correspond to the optical fibers 332 in FIGS. 3A-3C, the details of which are omitted for brevity. In the illustrated example, the endoilluminator 400 includes three optical fibers 432, although the endoilluminator 400 may include any suitable number (fewer or more than three) of optical fibers 432, for example, to provide desired illumination.

In the example of FIG. 4A, the pressure sensor 420 is positioned in an opening 426 on the sidewall of the tube 404 near the distal end 445 of the tube 404, and is connected to the electrical wire(s) 422 positioned in the longitudinal direction (e.g., the z-direction) along the optical fibers 432 in the bundle 424. In some embodiments, the pressure sensor 420 may substantially correspond to the pressure sensor 120 in FIG. 1.

FIG. 4B is a partial side perspective detailed view of a distal end portion 444 of the tube 404 shown in FIG. 4A. It is noted that the distal end portion 444 of the tube 404 may also be the distal end portion of the endoilluminator 400, and the distal end 445 of the tube 404 may also be the distal end of the endoilluminator 400. As illustrated in FIG. 4B, the bundle 424 includes the optical fibers 432 and electrical wire(s) 422 for the miniature pressure sensor 420.

As illustrated in FIG. 4B, a lens (or window) 460 is disposed in an opening 452 within a distal end 445 of the tube 404. The optical fibers 432 each terminate at or substantially near an interface 446 between the bundle 424 and the lens 460. The interface 446 and/or lens 460 may be configured to facilitate propagation of a desired illumination pattern from the optical fibers 432, onto a target site, such as a target site within an eye of a patient.

In some embodiments, a distal end 448 of the bundle 424 may abut a proximal end face 454 of the lens 460 at the interface 446 with positive pressure. In other implementations, one or more optically transmissive elements or materials may be situated at the interface 446 between the distal end face 448 and the lens 460. The lens 460 may substantially correspond to the lens 360 in FIGS. 3A-3C, the details of which are omitted for brevity.

FIG. 4C illustrates a cross-sectional view of the tube 404 of the endoilluminator 400 along the line 4C-4C as illustrated in FIG. 4B. As illustrated in FIG. 4C, the bundle 424 includes the optical fibers 432 disposed in an optional sleeve 434. In some embodiments, the sleeve 434 may include a filler material to fill in the space not occupied by the optical fibers 432. In some embodiments, the optical fibers 432 are coupled to a sidewall of the tube 404, or are freely disposed within the tube 404 without a sleeve.

In some embodiments, the optical fibers 432 may substantially correspond to the optical fibers 332 in FIGS. 3B and 3C. The details of the optical fibers 432 are omitted for brevity. Although three optical fibers 432 are shown in the illustrated example, the scope of the disclosure is not so limited. Rather, in other implementations, the endoilluminator 400 may include fewer optical fibers 432, while other implementations may include more than three optical fibers 432. In some implementations, the endoilluminator 400 may include two, four, or more optical fibers 432, and, in some examples, the optical fibers 432 may form a 2×2 array.

As illustrated in FIG. 4C, the pressure sensor 420 is disposed in the opening (or window) 426 on the sidewall of the tube 404 near the distal end 445. The opening 426 may extend through the entire thickness of the sidewall of the tube 404 to facilitate external exposure of the pressure sensor 420, thereby enabling direct contact with materials (e.g., fluid(s)) in the eye during ophthalmic procedures for direct IOP measurements. In certain examples, the pressure sensor 420 may interface (e.g., be in direct or immediate contact with) with intraocular space, such as with the materials in the eye to directly sense the IOP associated with the intraocular space in the eye. The electrical wire(s) 422 couple to the pressure sensor 420 within the tube 404. In some embodiments, the electrical wire(s) 422 may extend through at least a portion of the sleeve 434, and may be disposed along the longitudinal direction (e.g., z-direction) with the optical fibers 432. In some embodiments, as illustrated in FIG. 4C, the electrical wire(s) 422 may extend through the tube 404 external to the sleeve 434. In such embodiments, the electrical wire(s) 422 may extend through a second sleeve within the tube 404, and/or may be adhered to a sidewall of the tube 404.

Although a single pressure sensor 420 is shown in FIGS. 4B and 4C, the scope of the disclosure is not so limited. Rather, in other embodiments, the endoilluminator 400 may include more than one pressure sensor 420. In some implementations, the endoilluminator 400 may include one, two, four, or more pressure sensors 420. In the embodiments where multiple pressure sensors 420 are utilized, each of the pressure sensors 420 may be disposed in a separate opening (window) on the sidewall of the tube 404.

In certain embodiments, the tube 404 may have a diameter of about 1.5 mm or less, such as about 0.8 mm or less, although other suitable dimensions are also contemplated. In certain embodiments, the diameter of the tube 404 may be about 0.5 mm or less. Generally, the tube 404 may be sized to easily pass through a corresponding entry cannula, such as a trocar cannula, and into an intraocular space of a patient's eye. In certain embodiments, the diameter of the tube 404 corresponds to a gauge size of about 21-Ga, 22-Ga, 23-Ga, 24-Ga, 25-Ga, 26-Ga, 27-Ga, 28-Ga, 29-Ga, 30-Ga, or the like. In certain embodiments, the diameter of each pressure sensor 420 may be between about 0.25 to 1.55 mm, although other suitable dimensions are also contemplated.

Accordingly, the surgeon and/or the surgical console is provided a means for directly measuring IOP inside the eye, such that necessary adjustments (e.g., in response to a sudden change in IOP during surgery) can be made to more efficiently and safely control and/or maintain IOP in the eye.

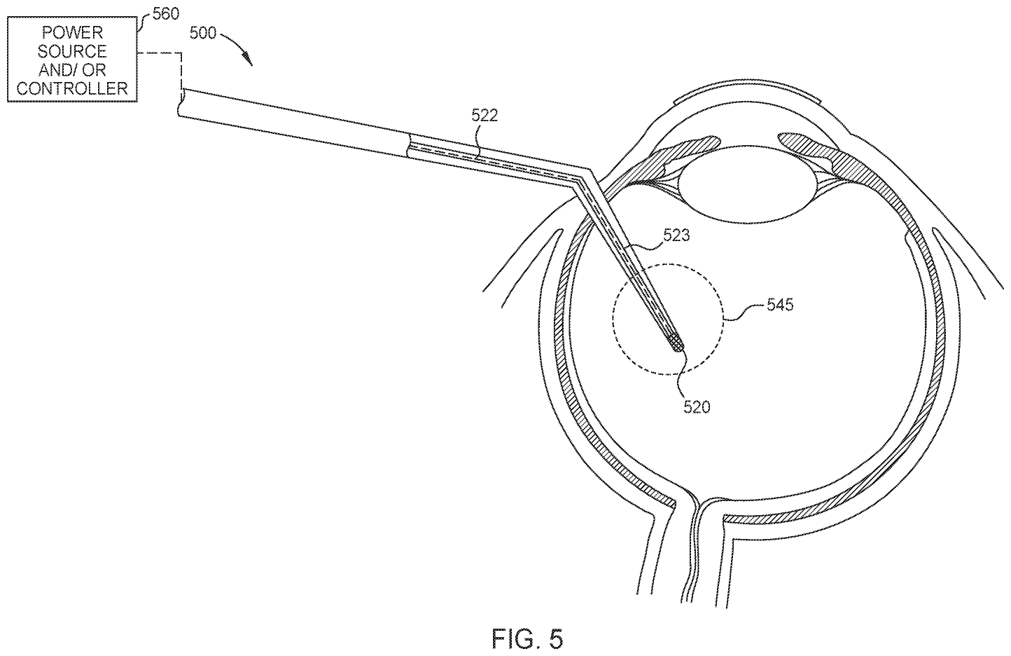

Although described above with reference to endoilluminators, the pressure sensing features presented herein are not limited to such devices. For example, the pressure sensing features presented herein may be integrated with other ophthalmic surgical instruments and/or devices inserted into the eye to provide substantially the same advantages as compared to conventional systems. Such ophthalmic surgical instruments and/or devices may include probes and other tools for performing procedures related to vitrectomy, keratoplasty, trabeculectomy, goniotomy, cataract surgery, and the like. Accordingly, FIG. 5 demonstratively illustrates a surgical instrument 500 having one or more pressure sensors 520 integrated at the distal end thereof, according to some embodiments of the present application.

As illustrated in FIG. 5, the pressure sensor(s) 520 may be integrated at a distal end portion 545 of the surgical instrument 500. The pressure sensor(s) 520 may be coupled to a power source and/or controller 560 (e.g., a power source and/or controller integrated in a surgical console) through one or more electrical wires 522 extending through an interior chamber 523 of the surgical instrument 500. In some embodiments, the pressure sensor(s) 520 may be coupled to electrical wires external to the surgical instrument 500. In some embodiments, the pressure sensor(s) 520 may be wirelessly powered by a battery pack, which may be integrated on the surgical instrument 500 (e.g., on the back end). In some embodiments, the pressure sensor(s) 520 may transmit and/or receive data/signals through a wireless transceiver.

In some embodiments, the surgical instrument 500 is configured to perform a surgical function. For example, the surgical instrument 500 may include a tool for manipulating ocular tissues during ophthalmic surgical procedures. Examples of tools for manipulating ocular tissues may include, but are not limited to, forceps, a spatula, a hook, a Sinskey hook, a depressor, and the like. In certain embodiments, the surgical instrument 500 may include a tool for cutting ocular tissues during ophthalmic surgical procedures. Examples of tools for cutting ocular tissues may include, but are not limited to, scissors, a side-port blade, a side-port knife, a knife for micro incision coaxial surgery (MICS), and the like. In certain embodiments, the surgical instrument 500 may include a probe for performing a particular type of ophthalmic surgery, such as a cutting probe, a vitrectomy probe, a phacoemulsification probe, a laser probe, an ablation probe, a vacuum probe, a flushing probe, and the like. In certain embodiments, the surgical instrument 500 may include a visualization device, such as an endoscopic visualization probe. In certain embodiments, the surgical instrument 500 includes an entry cannula, such as a trocar cannula. In certain embodiments, the surgical instrument 500 includes an infusion cannula. Still other ophthalmic devices, and/or combinations thereof, are contemplated.

While the distal end of the surgical instrument 500 is inserted into the eye during surgery to perform its surgical function, the pressure sensor(s) 520 may interface (e.g., be in direct or immediate contact with) with intraocular space, such as with the materials in the eye to directly sense the IOP associated with the intraocular space in the eye. Accordingly, the surgeon and/or the surgical console is provided a means for directly measuring IOP inside the eye, such that necessary adjustments (e.g., in response to a sudden change in IOP during surgery) can be made to more efficiently and safely control and/or maintain IOP in the eye.

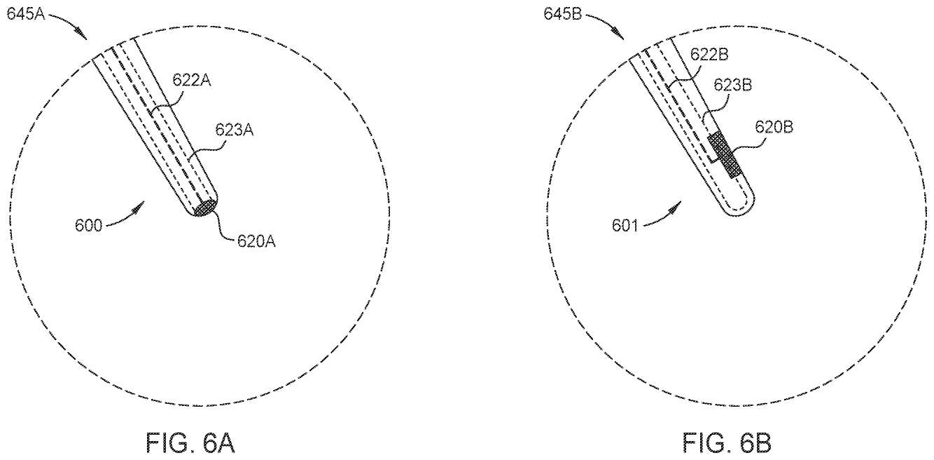

FIG. 6A illustrates a distal end portion 645A of a surgical instrument 600, according to some embodiments of the present disclosure. In some embodiments, the surgical instrument 600 may substantially correspond to the surgical instrument 500 in FIG. 5. In some embodiments, the distal end portion 645A may correspond to the distal end portion 545 in FIG. 5. As illustrated in FIG. 6A, a pressure sensor 620A is integrated at the distal tip of the surgical instrument, and coupled to electrical wire(s) 622A embedded in an interior chamber 623A of the surgical instrument.

FIG. 6B illustrates a distal end portion 645B of a surgical instrument 601, according to some embodiments of the present disclosure. In some embodiments, the surgical instrument 601 may substantially correspond to the surgical instrument 500 in FIG. 5. In some embodiments, the distal end portion 645B may correspond to the distal end portion 545 in FIG. 5. As illustrated in FIG. 6B, a pressure sensor 620B is integrated in an opening on a sidewall of the surgical instrument, and coupled to electrical wire(s) 622B embedded in an interior chamber 623B of the surgical instrument.

FIG. 7 illustrates a schematic diagram of a surgical console 740, according to embodiments disclosed herein. In some embodiments, the surgical console 740 may substantially correspond to the surgical console 140 of FIG. 1.

As shown in FIG. 7, the surgical console 740 includes, without limitation, a control module 762, a user interface 764, an interconnect 766, and at least one I/O (Input/Output) device interface 768, which may allow for the connection of various I/O devices (e.g., keyboards, displays, mouse devices, pen input, etc.) to the surgical console 740. The surgical console 740 may also include an illumination source 708 (e.g., a continuous illumination source and/or a stroboscopic illumination source) coupled to optical fibers 732 of an endoilluminator (e.g., the endoilluminator 100 of FIG. 1) through a cable 710a. In some embodiments, the surgical console 740 may operatively couple to an illumination source (e.g., a continuous illumination source and/or a stroboscopic illumination source) outside of the surgical console 740. The surgical console 740 may further include one or more pressure management tools 786 for managing IOP in the eye, for example, through venting and/or controlling the rate of infusion and/or aspiration. In some embodiments, the pressure management tools 786 include pumps, vacuums, and/or other devices for controlling infusion and/or aspiration, for example, via a probe that may be inserted in the patient's eye. The surgical console 740 may, in some embodiments, include a display in the user interface 764 for displaying information to a user, including surgical procedural parameters and device settings (the display may also incorporate a touchscreen for receiving user input).

The control module 762 includes a processor 714 (e.g., a controller), a memory 770, and a storage 772. The processor 714 (e.g., control circuitry) is configured to retrieve and execute programming instructions stored in the memory 770. Similarly, the processor 714 may retrieve and store application data residing in the memory 770. The interconnect 766 transmits programming instructions and application data, among the processor 714, I/O device interface 768, user interface 764, memory 770, storage 772, illumination source 708, pressure management tool(s) 786, etc. The processor 714 may include a single CPU (Central Processing Unit), multiple CPUs, a single CPU having multiple processing cores, and the like. The memory 770 may be random access memory, and the storage 772 may be a disk drive. Moreover, the memory 770 and/or storage 772 may be any type of a readily available memory, such as random access memory (RAM), read only memory (ROM), floppy disk, hard disk, solid state, flash memory, magnetic memory, or any other form of digital storage, local or remote. In certain embodiments, the memory 770 and/or storage 772 include instructions, which when executed by the processor 714, cause the pressure management tool(s) 786 to manage (e.g., adjust, control, increase, decrease, etc.) the IOP in a patient's eye based on direct IOP measurements. For example, memory 770 includes a pressure management module 784, which may include computer-executable instructions that, when executed by the processor 714, cause the processor 714 to control the pressure management tool(s) 786 in order to manage the IOP in the patient's eye based on IOP measurements reflected by signals received from the one or more pressure sensors 720.

In particular, the processor 714 may receive signals from one or more pressure sensors 720 through electrical wiring 710b. These signals, for example, correspond to readings of fluidic pressure (e.g., IOP) in the eye. The processor 714 is also configured to send output signals via the interconnect 766 to the pressure management tool(s) 786. For example, these output signals allow the processor 714 to control the operations of the pressure management tool(s) 786 (e.g., venting valves, output valves, infusion and aspiration valves and pumps, vacuum sources, etc.) based on the signals from the one or more pressure sensors 720 for maintaining or controlling the intraocular pressure of the eye.

In the embodiment of FIG. 7, the processor 714 may include an integrated circuit capable of performing logic functions. In this manner, the processor 714 is in the form of a standard integrated circuit package with power, input, and output pins. In various embodiments, the processor 714 may control one or more valve or pump controllers, or other targeted device controllers. In such a case, the processor 714 may perform specific control functions targeted to a specific device, such as one or more valve(s), infusion pumps, and/or aspiration pump(s). In other embodiments, the processor 714 is a microprocessor. In such a case, the processor 714 is programmable so that it can function to control valve(s) and infusion and/or aspiration pump(s) as well as other components of coupled to the surgical console. In other cases, the processor 714 is not a programmable microprocessor, but instead is a special purpose controller configured to control different valve(s) and pump(s) that perform different functions.

The surgical console 740 may be configured to drive one or more surgical instruments 790, which may include ophthalmic probes of a variety of probe types, including endoilluminators, laser probes (e.g., picosecond infrared laser probes, femtosecond laser probes), vitrectomy probes, phacoemulsification probes, flap cutters, and other ophthalmic surgical tools. In operation, the surgical console 740 may function to assist a surgeon in performing various ophthalmic surgical procedures, such as vitrectomy, phacoemulsification, cataract surgery, LASIK, and similar procedures. In some embodiments, the surgical instruments 790 may also include one or more valves and infusion pumps and lines.

In embodiments where the surgical instrument(s) 790 include a vitrector, the surgical console 740 may include one or more modules or components to power the vitrector for the purpose of breaking-down (e.g., cutting) the vitreous. For example, in certain embodiments, the surgical console 740 may include a pneumatic module that uses compressed gas, such as nitrogen, to power the vitrector. In certain other embodiments, the surgical console 740 may include a laser source for generating laser light that is used by the vitrector to break-down the vitreous (for example, see U.S. Patent Publication No. 2019/0201238).

In some embodiments, the surgical tool(s) 790 may include a phacoemulsification probe. For example, the surgical instrument(s) 790 may include an ultrasonic phacoemulsification probe that is capable of emulsifying or breaking-down a lens during cataract surgery. As another example, the surgical instrument(s) 790 may be configured to emit laser light for lens emulsification. In embodiments where the surgical instrument(s) 790 may be a phacoemulsification probe, the surgical console 740 includes one or more modules or components to power the phacoemulsification probe to emulsify the lens during cataract surgery. In some embodiments, the surgical instrument(s) 790 may include a picosecond infrared laser (pIRL).

In some embodiments, the surgical instrument(s) 790 may be configured to emit laser light, such as femtosecond laser light, used to make incisions and/or cut flaps during ophthalmic surgery. A suitable example femtosecond laser is a WaveLight® F S200 laser available from Alcon, Inc. of Fort Worth, Texas. In some embodiments, the surgical instrument(s) 790 may be a laser, for example an excimer laser for photorefractive keratectomy and/or LASIK procedures (e.g., laser ablation of the cornea). A suitable example excimer laser is a WaveLight® EX500 laser available from Alcon, Inc. of Fort Worth, Texas.

In some embodiments, the surgical instrument(s) 790 may include an endoilluminator. In such embodiments, the optical fibers 732 may be disposed through the endoilluminator for introduction into the eye of the patient to propagate light from the illumination source(s) 708 thereto.

In some embodiments, one or more of the pressure sensors 720 may be integrated into a distal portion of at least one of the surgical instrument(s) 790. Accordingly, this facilitates the positioning of the pressure sensors 720 in the eye to directly sense the IOP associated with the intraocular space in the eye. In some embodiments, the one or more pressure sensors 720 may interface (e.g., in direct or immediate contact with) with intraocular space, such as with the materials in the intraocular space via an opening in the distal portion of the at least one surgical instrument 790.

The surgical instrument(s) 790 may be operatively coupled to the surgical console 740 via one or more ports of the surgical console 740. It is noted that surgical instrument(s) 790 may be operatively coupled to the surgical console 740 via a number of different tubes or cables configured to interface with ports of the surgical console 740. For example, such tubes or cables may include a pneumatic line, an optical fiber cable, an ultrasound power line for powering the surgical instrument(s) 790 for cutting purposes, as well as an aspiration or vacuum line for transporting the aspirated matter back to the surgical console 740.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims. Those skilled in the art will appreciate that the surgical instruments (e.g., endoilluminators and auxiliary surgical instruments) illustrated herein can include more components than the simplified illustrations described herein. The surgical instruments described herein include only those components useful for describing some prominent features of implementations within the scope of the claims.

What is claimed is:

1. An endoilluminator, comprising:
a handpiece; and
a probe coupled to a distal end of the handpiece, the probe comprising an optical fiber and a pressure sensor, wherein:
the pressure sensor is positioned near a distal end of the probe,
the pressure sensor is configured to be inserted into an intraocular space of an eye, and
the pressure sensor is configured to directly sense an intraocular pressure (IOP) associated with the intraocular space of the eye.

2. The endoilluminator of claim 1, wherein the pressure sensor comprises at least one of a microelectromechanical system (MEMS) pressure sensor, a piezoelectric pressure sensor, a potentiometric pressure sensor, an inductive pressure sensor, a strain gauge pressure sensor, and a capacitive pressure sensor.

3. The endoilluminator of claim 1, wherein:
the distal end of the probe comprises an opening through which light from a distal end of the optical fiber is configured to be propagated; and
the pressure sensor is configured to sense the IOP through the opening.

4. The endoilluminator of claim 1, wherein:
the distal end of the probe comprises a first opening through which light from a distal end of the optical fiber is configured to be propagated; and
the distal end of the probe comprises a second opening through which the pressure sensor is configured to sense the IOP.

5. The endoilluminator of claim 4, wherein the second opening is disposed in a sidewall of the probe adjacent the distal end.

6. The endoilluminator of claim 1, wherein the pressure sensor is coupled to one or more electrical wires running substantially along a longitudinal direction of the optical fiber in the probe.

7. The endoilluminator of claim 1, wherein the endoilluminator is coupled to a light source configured to drive light into the optical fiber.

8. The endoilluminator of claim 1, wherein the endoilluminator is coupled to a surgical console providing electrical power to the pressure sensor.

9. An instrument for ophthalmic surgery, the instrument comprising:
a pressure sensor positioned near a distal end of the instrument, wherein the pressure sensor is configured to:
be inserted into an intraocular space of an eye, and
directly sense an intraocular pressure (IOP) associated with the intraocular space of the eye.

10. The instrument of claim 9, further comprising: a power source coupled to the instrument for providing electrical power to the pressure sensor.

11. The instrument of claim 9, wherein the pressure sensor comprises at least one of a microelectromechanical system (MEMS) pressure sensor, a piezoelectric pressure sensor, a potentiometric pressure sensor, an inductive pressure sensor, a strain gauge pressure sensor, and a capacitive pressure sensor.

12. The instrument of claim 9, wherein the pressure sensor is disposed at a tip or a sidewall of the distal end of the instrument.

13. The instrument of claim 9, wherein the instrument comprises at least one of a pair of forceps, a spatula, a hook, or a depressor.

14. The instrument of claim 9, wherein the instrument comprises at least one of a pair of scissors or a knife.

15. The instrument of claim 9, wherein the instrument comprises at least one of a vitrectomy probe, a phacoemulsification probe, a laser probe, a vacuum probe, an endoscopic visualization probe, or an endoilluminator.
